(19) United States
(12) Patent Application Publication
(10) Pub. No.: US 2009/0018470 A1
(43) Pub. Date: Jan. 15, 2009

(54) DEVICE

(76) Inventors: Ara W. Darzi, Buckinghamshire (GB); Edward H. Goodwin, London (GB); Lee D. Edwards, Essex (GB); Omer Aziz, Surrey (GB); Richard A. Hartshorn, London (GB)

Correspondence Address: DICKSTEIN SHAPIRO LLP, 1825 EYE STREET NW, Washington, DC, 20006-5403, US

(21) Appl. No.: 12/067,623

(22) PCT Filed: Sep. 21, 2006
(86) PCT No.: PCT/GB2006/003533

§ 371 Date: Sep. 30, 2008

(30) Foreign Application Priority Data
Sep. 21, 2005 (GB) 0519259.6

(51) Int. Cl. A61B 5/01

(52) U.S. Cl. 600/593

(57) ABSTRACT

A device (10) for causing dilation of a tubular anatomy comprising a distal end and a proximate end, and: an active element (100) comprising: a flexible portion (110) expandable between a first, non expanded state, and a second, expanded state; an activator (40) for causing expansion of the flexible member; and a pressure sensor (200) operably connected to the flexible member.

This invention relates to a device for causing dilation, and measuring pressure within a tubular anatomy, and particularly, but not exclusively, to a device for measuring pressure within the oesophagus of a human or animal.

The oesophagus is a tube that connects the mouth with the stomach. The walls of the oesophagus are very muscular, and contract rhythmically. This rhythmical movement is known as peristalsis and serves to transfer food from the mouth to the stomach for digestion within the stomach.

The oesophagus is connected to the stomach by a valve known as the lower oesophageal sphincter. The sphincter prevents the backward flow of food from the stomach into the oesophagus.

It can sometimes be necessary to measure how well the muscles of the oesophagus are working and to measure the strength of the lower oesophageal sphincter. Such measurements may assist diagnosis of a medical condition present in a patient.

These measurements are known as oesophageal manometry or as an oesophageal function or oesophageal motility study.

It is known to carry out such measurements by passing a soft tube through the nose or mouth of a patient. The tube has pressure sensors along its length and when in place can measure the pressure that is produced by the oesophageal muscles when relaxing or compressing during the peristaltic process, or the pressure within a stricture.

It is known that strictures may develop in the oesophagus. The presence of one or more strictures in an oesophagus may lead to problems and clinical conditions such as dyspepsia, dysphasia and asthma. It is therefore desirable to be able to dilate the oesophagus in the region of the stricture in order to reduce or overcome these problems.

It is known to use a device which is generally olive shaped and made of metal to dilate the oesophagus in the region of a stricture. Such devices are available in a range of sizes, and are passed through the stricture in turn, starting with the largest device that can just pass through the stricture, and increasing the size of the device until the desired dilation of the stricture is achieved. It is also possible to use, in a similar way, soft, flexible rubber tubes in place of the metal olive shaped devices.

Surgery of the oesophagus, particularly conventional or laparoscopic fundoplication used in the treatment of gastroesophageal reflux disease can result in severe post-operative dysphagia. This is because the wrap (i.e the stomach wall wrapped around the oesophagus) is too tight. The ability to perform manometry in the oesophagus pre, intra and post operation would reduce the extent of dysphagia suffered since it would enable the wrap tension to be measured.

In addition, a means for carrying out pre, intra and post operative manometry combined with a means for measuring the dilation of strictures would assist in the selection of the most appropriate technique to use to carry out the surgery.

According to a first aspect of the present invention there is provided a device for causing dilation of a tubular anatomy comprising a distal end and a proximate end, and:
an active element comprising:
a flexible portion expandable between a first, non-expanded state, and a second, expanded state;
an activator for causing expansion of the flexible portion; and
a pressure sensor operably connected to the flexible portion.

According to a second aspect of the present invention there is provided a device for causing dilation of a tubular anatomy comprising a distal end and a proximate end, and:

an active element comprising:

a flexible portion expandable between a first, non-expanded state, and a second, expanded state;

an activator for causing expansion of the flexible member;

wherein:

the flexible portion comprises a plurality of elongate flexible members each having a first end positioned towards the distal end of the device, and a second end positioned towards the proximate end of the device, wherein the activator is adapted to cause circumferential movement of the distal end of the device relative to the proximate end of the device.

Advantageously, the activator is adapted to cause circumferential rotation of the distal end in a first sense, and to cause circumferential rotation of the proximate end in an opposite sense.

A device according to the present invention may be positioned within a tubular anatomy such as the oesophagus whilst the flexible member is in its first, non-expanded state. The activator may then be used to cause the flexible portion to expand into an expanded state.

Because the pressure sensor is operably connected to the flexible portion, rather than to any other part of the device, it is possible to accurately measure pressure within the tubular anatomy at any given time. Pressure data may be transmitted to a user by any desirable means. For example, the pressure data may be transmitted as an electrical signal and displayed via a display unit such as a digital display. The signal may also be inputted to a computer for analysis, collection and display.

The activator is controlled to exert a predetermined pressure on the flexible portion. This means that an appropriate degree of dilation may be achieved.

The pressure applied by the activator may be either constant or stepped to allow a gradual expansion of predetermined dilation sizes. It is thus possible to achieve controlled dilation of a stricture in the tubular anatomy.

The pressure sensor may be any type of pressure sensor, but advantageously the pressure sensor comprises a strain gauge.

The pressure sensor may also comprise a capacitive pressure sensor, TiN pressure sensor, or piezoelectric polymer sensors, for example.

Alternatively, the pressure sensor comprises a sensor skin mounted on an outer surface of the flexible portion. Such a pressure sensor will provide independent pressure measurement of the pressure existing in the tubular anatomy independent of the pressure exerted on the device to maintain the device in its expanded state.

Preferably, the flexible portion comprises a plurality of elongate flexible members each having a first end positioned towards the distal end of the device, and the second end positioned towards the proximate end of the device, the device further comprising a first connector connected to the first ends of the members, and a second connector connected to the second end of the members.

Advantageously, the activator comprises a compressor for causing one of the connectors to move towards the other connector, thereby causing the elongate members to bow laterally.

Thus, in an embodiment of the device comprising a plurality of elongate flexible members, expansion of the device will expand radially and compress axially.

In one embodiment of the invention, the activator causes both radial and axial expansion of the flexible portion.

The device may comprise a plurality of flexible portions connected to one another in a modular fashion. This enables the overall length of the device to be increased while enabling the size of each flexible portion to remain relatively short. The dimensions of the flexible portions will depend on the use to which the device is to be put.

In another embodiment of the invention, the flexible portion is enclosed within a passive outer shell. Expansion of the flexible member causes corresponding expansion of the passive outer shell. Due to the presence of the flexible portion and the passive outer shell, the device has a greater structural strength than a device where no outer shell is present.

Advantageously, the pressure sensor is positioned on one of the elongate flexible members. By positioning the pressure sensor on one of the elongate members, it is possible to obtain an accurate measurement of the pressure within the tubular anatomy.

Advantageously, the pressure sensor is defined by one or more cuts formed in the elongate flexible member. The one or more cuts in the flexible elongate member serve to substantially isolate the pressure sensor from the device thus allowing the pressure sensor to take pressure measurements of the tubular anatomy independent of any pressure exerted on the elongate member by the activator, to cause expansion of the device.

Advantageously, the device comprises a plurality of pressure sensors, each of which sensors is operably connected to an elongate flexible member.

By having a plurality of pressure sensors, it is possible to obtain pressure readings at different points within the tubular anatomy. This enables a user to obtain more accurate information relating to the pressure existing within the tubular anatomy.

By means of the present invention, therefore, it is possible to achieve dilation of a stricture in a tubular anatomy without having to use a plurality of devices each having a predetermined size. This in turn means that the device does not have to be taken in and out of the tubular anatomy, since once the device has been inserted, it can remain in place until the surgical operation or procedure is complete. This reduces the severity of dysphagia experienced after the operation or procedure.

Advantageously, the device further comprises at least one stop for preventing over-compression of the active element. This ensures that over dilation does not occur. The expansion of the active element is restricted by the at least one stop, past which the moveable elements cannot move. In other words, maximum expansion is set.

Preferably, the device comprises a plurality of stops positioned to ensure stepped dilation of the tubular anatomy. The stops may comprise a plurality of grooves positioned along the length of the active element and a moveable member in the form of a ring or disc. The grooves are adapted to retain the moveable ring and are each collated with a predetermined movement of the elongate members.

Alternatively, the device comprises a screw thread extending along the length of the active element, and a moveable member, moveable along the screw thread.

Conveniently, the moveable member is internally threaded.

The moveable member is moveable along the screw thread. This means that the movement of the elongate members is not limited to predetermined positions defined by the position of grooves, for example. This in turn means that dilation of the active element is not stepped.

Conveniently the first and second connectors are substantially disc shaped. The device in its un-bowed state is therefore substantially cylindrical in shape. This ensures easy insertion into a tubular anatomy.

The connectors may be formed integrally with the elongate members. In other words, the elongate members and the connectors may be formed from a unitary sheet. Alternatively, the connectors may be formed separately from the elongate members.

Advantageously, the compressor comprises a first sleeve associated with the active element, which sleeve is mechanically driven. When activated, the sleeve pushes against one end of the active element causing compression of the active element.

Alternatively, the compressor comprises means for pulling one connector towards the other. Conveniently, the compressor comprises a wire connected to the distal end of the device. By pulling the wire it is possible to cause the distal end of the device to move towards the proximate end of the device.

Conveniently, the compressor comprises a threaded elongate member associated with the active element.

In an alternative embodiment, the flexible member comprises a balloon. The device further comprises a plurality of pockets formed on an outer surface of the balloon, each of which pockets contains a fluid, the device further comprising a pressure sensor operatively connected to each pocket. The fluid may be, for example, water, oil or another incompressible fluid that will directly transfer the increase in pressure to a measuring device.

In use, the pressure generated in the fluid within the pockets will be independent of the pressure exerted on the device in order to maintain the device in an expanded state. The pressure sensors associated with each of the pockets may be mounted elsewhere in the device.

Advantageously, the device further comprises an outer sleeve extending over the active element. This allows smooth passage of the device through the tubular anatomy. The outer sleeve may be made from any suitable material, but preferably it is made from rubber.

Alternatively, the outer sleeve may be made from polytetrafluoroethylene or a polyurethane elastomer.

Other parts of the device, such as the flexible members may be made from any suitable material such as Nitinol (Nytinol), or steel.

Preferably, the device further comprises a cone at the distal end of the active element. The cone facilitates initial passage though a stricture and reduces or prevents damage to the surrounding tissue and structure of the tubular anatomy.

Conveniently, the cone is made from rubber.

According to a third aspect of the present invention there is provided a method of dilating a tubular anatomy, the method comprising inserting a device for causing dilation of a tubular anatomy into the tubular anatomy to be dilated, the device comprising:

an active element comprising:
a flexible portion expandable between a first, non-expanded state, and a second, expanded state;
an activator for causing expansion of the flexible portion; and
a pressure sensor operably connected to the flexible portion;

the method comprising:
inserting the device into a tubular anatomy causing expansion of the flexible portion;
measuring the pressure with the tubular anatomy, collapsing the device, and
removing the device from the tubular anatomy.

The invention will now be further described by way of example only with reference to the accompanying drawings in which:

FIG. 1 is a schematic representation of a device according to the present invention;

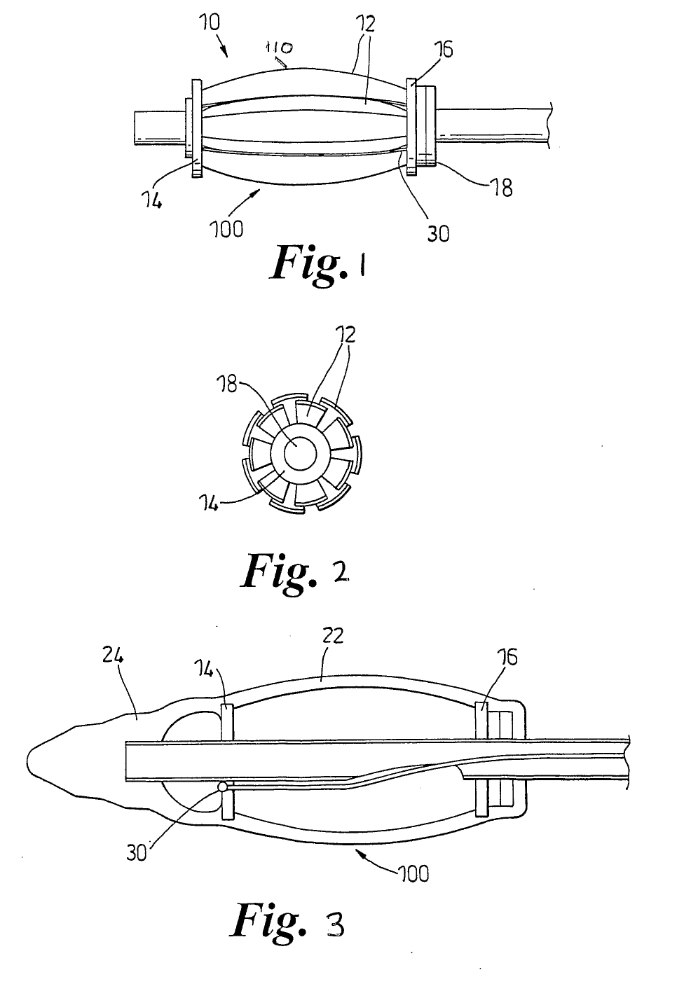

FIG. 2 is a cross-sectional representation of the device of FIG. 1;

FIG. 3 is a schematic representation of the device of FIG. 1 with the outer sleeve in place;

FIG. 4 is a schematic representation of the device of FIG. 1 in a non bowed stated;

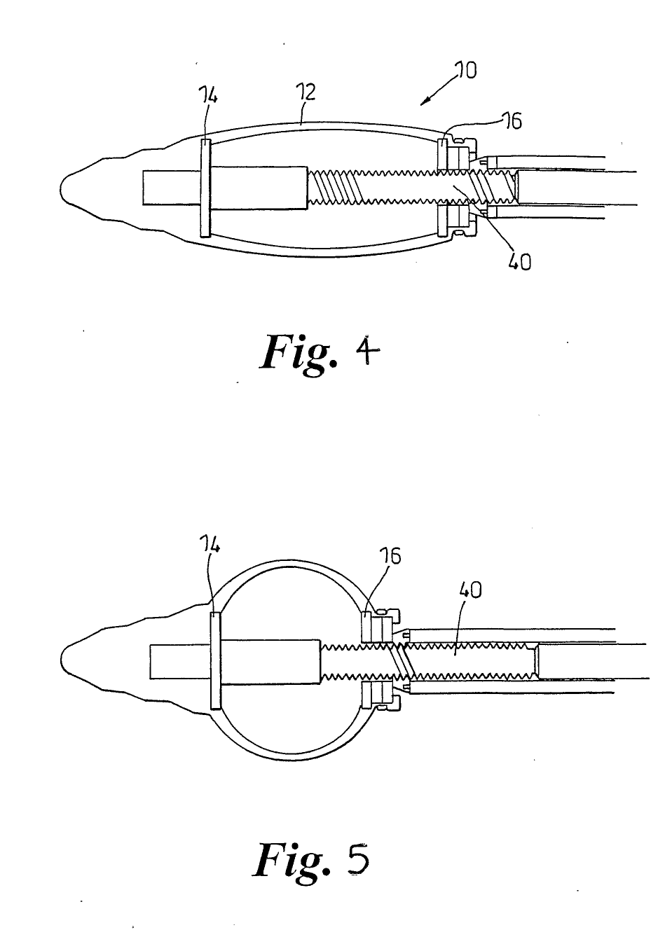

FIG. 5 is a schematic representation of the device of FIG. 1 in a bowed state;

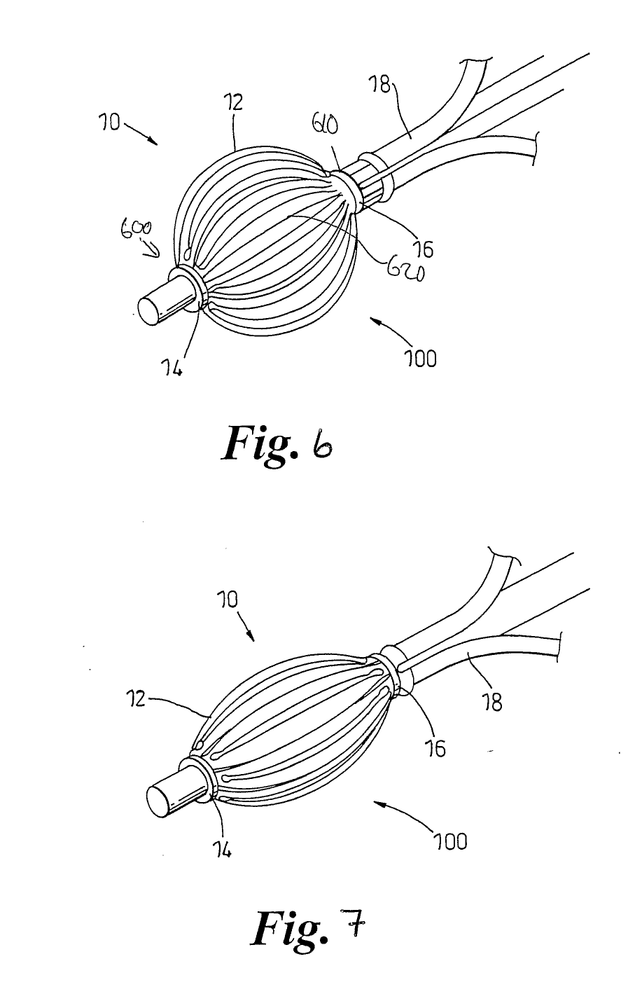

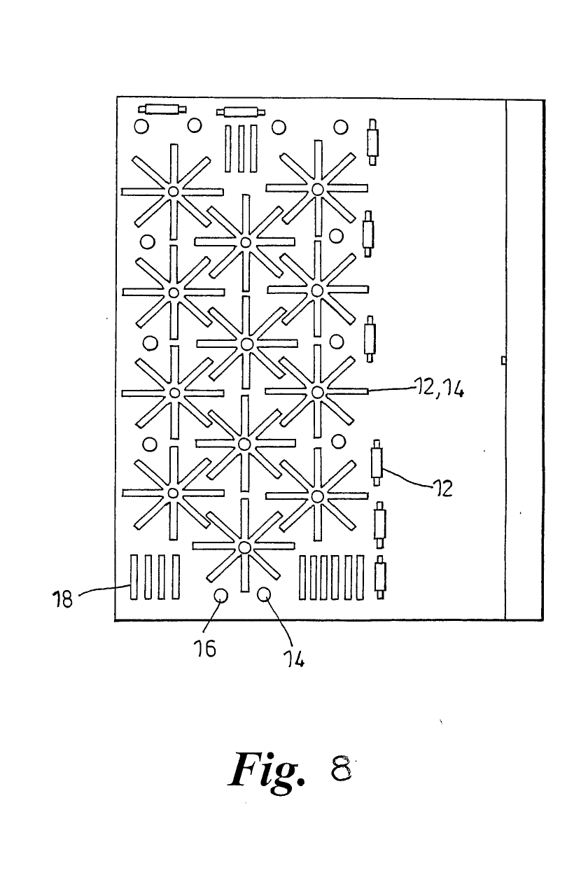

FIGS. 6 and 7 are schematic representations of a second embodiment of a device according to the present invention;

FIG. 8 is an illustration of components forming a plurality of devices according to an embodiment of the invention;

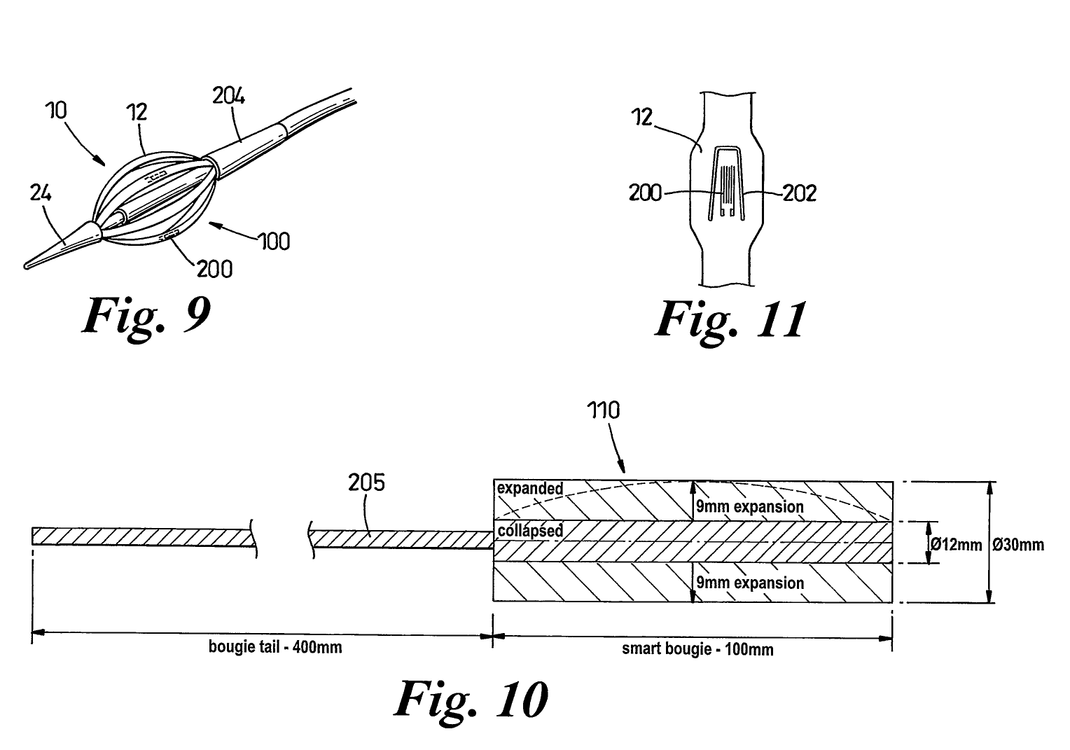

FIG. 9 is a schematic representation of a third embodiment of a device according to the present invention in which pressure sensors are mounted on one or more of the elongate members;

FIG. 10 is a schematic representation showing graphically expansion of the device of FIG. 9;

FIG. 11 is a schematic representation showing in more detail a pressure sensor forming part of the device of FIG. 9;

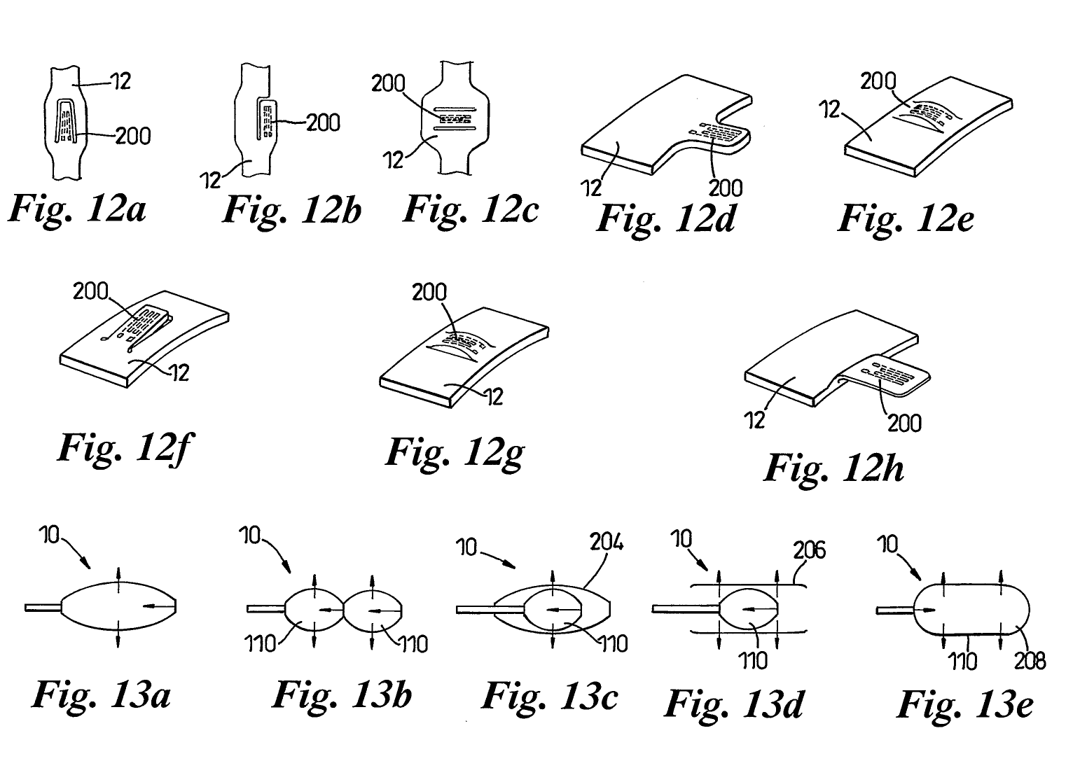

FIGS. 12a to 12h are schematic representations showing further possible configurations of the pressure sensor forming part of the device of FIG. 9;

FIGS. 13a to 13e are schematic representations of further possible configurations of a device according to the present invention; and

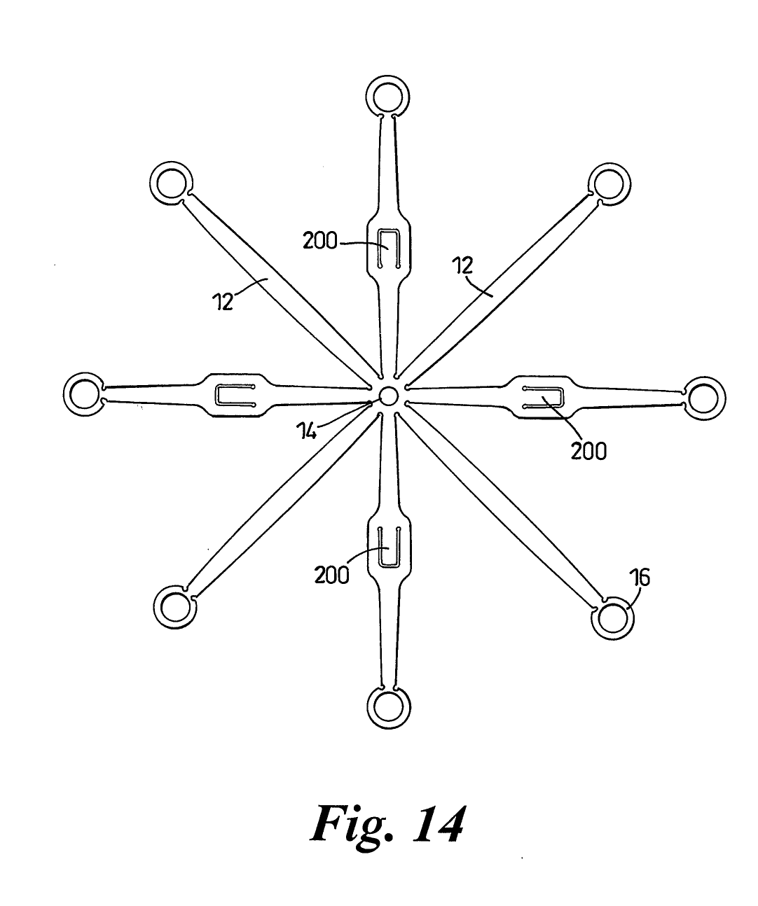

FIG. 14 is an illustrations of components forming the device of FIG. 9.

Referring to the figures, and initially to FIGS. 1 to 8, a device according to an embodiment of the present invention is designated generally by the reference numeral 10. The device can be used in, for example, an oesophageal manometry and also for the measurement of dilation within a tubular anatomy such as the oesophagus. The device may also be used to dilate strictures within the oesophagus.

The invention will be described in terms of use within oesophagus of a human or animal. However, it would also be a great benefit in a wide range of surgical procedures of tubular anatomy including the oesophagus; ureter, urethra, bronchus, or similar tubular structures; and vascular and cardiac structures.

The device according to the present invention could also be used for expandable dilation and debridement in coronary and peripheral arterectomy. The device according to the present invention may also find application in orthopedic surgical debridement.

The invention may also be used in balloon angioplasty procedures.

An embodiment of the invention comprises a device 10 comprises a flexible portion 110 comprising a plurality of elongate members 12 in the form of flexible strips. The elongate members 12 may be made from any convenient material for example metal. The flexible strips are held by connectors 14, 16 which, in the non active state hold the elongate members 12 in a substantially cylindrical shape. The connectors 14, 16 together with the strips 12 comprise the active element 100 of the device 10.

Although in this example the connectors 14, 16 are shown as being formed separately from the elongate members 12, in other embodiments, the connectors 14, 16 may be formed integrally with the elongate members 12.

The device further comprises a first sleeve 18 which is mechanically driven. By driving the sleeve 18 towards connector 14, the elongate members 12 are caused to bow out laterally as shown in FIG. 1. The sleeve 18 may be controlled by any suitable means for example it may be computer controlled.

By means of the expansion of the elongate members 12, predetermined stages of dilation of the oesophagus, particularly a stricture in the oesophagus may be achieved.

By means of the present invention therefore it is not necessary to have a range of devices of graduated size and to repeatedly insert appropriately sized devices into a patient's oesophagus. It is merely necessary to insert the device according to the invention once, and to cause dilation of the stricture in a controlled manner.

The expansion of the elongate members 12 may take place either continuously with a gradual increase in expansion, or may take place in stepped increases, depending on the conditions prevailing.

The sleeve 18 may be moved by the tensioning or pulling of an integral wire 30 connected to the distal end of the connector 14. Alternatively, a wire may be connected to the connector 16 (FIG. 3).

The active element 100 is covered with an outer sleeve 22 (FIG. 3) which allows smooth passage of the device 10 through an oesophagus or similar tubular anatomy.

The device further comprises a cone 24 fitted to the distal end of the device which facilitates initial passage through a stricture and prevents or reduces damage to surrounding tissue and structure. The cone may be made of soft rubber, or any other suitable material.

The device 10 is designed so that it can either form a component of an existing device, such as a flexible fibre optic endoscope, an endoscope insertion tube, a non-viewing endoscope tube; or it may be a stand alone device.

Referring to FIGS. 4 and 5, the device 10 is shown to comprise a compressor 40 in a form of a mechanical compression screw. The connector 14 is attached to the screw 40, and by tightening the screw 40, the connector 14 is moved towards the connector 16. This causes the elongate members 12 to bow and to take the position shown in FIG. 5.

Referring to FIGS. 6 and 7, a further embodiment of a device 10 according to the present invention is shown. Parts of the device 10 which correspond to parts of the device 10 as illustrated in FIGS. 1 to 5 have been given corresponding reference numerals for ease of reference.

FIG. 6 shows the device 10 in an active, or dilated state, and FIG. 7 shows the device in a non-active state.

In a further embodiment of the device shown in FIGS. 6 and 7. The device further comprises means (not shown) for causing circumferential rotation of one end 600 of the device 10 in addition to longitudinal compression of the device.

If one end 600 of the device 10 is circumferentially rotated whilst a second end 610 is held fixed, and the device is simultaneously axially compressed, the flexible members 12 will splay such that a leading edge 620 of each flexible member will protrude from the device and may thus act as a blade-like structure. The same effect can be achieved if one end of the device 610 is rotated circumferentially in a first sense, and another end of the device is rotated circumferentially in an opposite sense.

The entire device may then also be rotated and the resulting device may act as a cutter/debrider.

Such a device may also comprise an internal suction tube (not shown) which could be used to remove debris loosened through the cutting/debriding action of the device 10.

Such a device could not only be used to dilate a tubular anatomy, but could also be used to debride such a tubular anatomy.

Such a device would have application in coronary and peripheral arterectomy.

In any artery where calcified plague has lined the internal walls of the artery, a further device, for example in the form of a diamond tipped high speed rotating device could follow the device 10 in order to remove plague after the device 10 has been moved through the artery.

Referring to FIG. 8, components which may be used to form a plurality of the devices shown in FIGS. 8 and 9 are illustrated. The illustrated components comprise a plurality of flexible members 12, connectors 14, 16 and sleeves 18.

A further embodiment of the device 10 according to the present invention will now be described with reference particularly to FIGS. 9 to 14. Parts of the device 10 corresponding to parts of embodiments of the invention described hereinabove have been given corresponding reference numerals for ease of reference. The device 10 comprises a plurality of flexible elongate members 12. Attached to one or more of the elongate members 12 is a pressure sensor 200 in the form of a strain gauge mounted on a flexible elongate member 12. The strain gauge may be defined within the elongate member 12 by means of one or more cuts 202 as shown in FIG. 11. The cuts 202 serve to isolate the strain gauge thus allowing pressure measurements to be taken within a tubular anatomy that are substantially independent of any pressure exerted on the device in order to maintain the device in an expanded state.

The device shown in FIGS. 9 and 10 comprises an active element 100 having a length in a collapsed state of approximately 100 mm. The diameter of the active element in the collapsed state is approximately 12 mm, and in the expanded state is approximately 30 mm.

The device could, however, have desirable dimensions. For example, the active element could have a diameter of approximately 3 mm in the collapsed state.

The device 12 further comprises a front cone 24 and a tail cone 204 both of which are formed from a flexible material in order to allow the device to be able to pass down a tubular anatomy such as the oesophagus. The length of the tail cone 204 is approximately 20 mm, and the total length of a tail portion 205 connecting the device to a display unit, for example, is approximately 400 mm.

The dimensions of the device 9 are shown graphically in FIG. 10. It is to be understood however that the device according to the present invention could have any desirably dimensions.

The pressure sensor could have a number of different configurations as shown in FIG. 12a which shows a pressure sensor 200 mounted on a flexible element 12.

The pressure sensor may be orientated appropriately to measure the expected pressures within the tubular anatomy.

For example, as shown in FIG. 12c, the pressure sensor 200 may be orientated so that the direction of strain sensing is orthogonal to the direction of strain in the flexible member 12.

As shown in FIGS. 12d, 12e, 12f and 12h, the strain gauge 200 may protrude from the structural member to ensure that the sensor touches the wall of the tubular anatomy when the device is in situ within the anatomy.

As shown in FIGS. 12e, 12g and 12h, the strain gauge 200 may be etched so that it has a thickness that is less than the thickness of the flexible member 12 on which it is mounted. This results in a greater sensitivity of the pressure sensor.

Referring now to FIGS. 13a to 13e, further embodiments of a device 10 are illustrated schematically.

FIG. 13a is a schematic representation of the device of FIG. 9.

FIG. 13b is a schematic representation of a device 10 comprising two flexible portions 110 attached together. The flexible portions 110 may be attached together in a modular manner, and any number of flexible portions 110 may be so attached.

In FIG. 13c, a device 10 is illustrated in which the flexible portion 110 is encased within a passive outer shell 204. The flexible portion is used to expand the device 10, and the presence of the passive outer shell 204 provides additional structural strength to the device 10. The outer shell may be formed from any suitable material such as metal, rubber or plastic.

FIG. 13d illustrates a device 10 having an outer casing 206 which results in linear expansion of the device 10.

FIG. 13e represents schematically a device 10 comprising a flexible portion formed from a balloon 208.

Turning now to FIG. 14, the components used to form the flexible elements of the device 10 shown in FIG. 9 are illustrated schematically.

When the device 10 forms part of an existing device such a flexible fibre optic endoscope, the endoscope may be positioned within a oesophagus in order to view a stricture. The device 10 may then be passed through an operative channel in the endoscope and positioned at the site of the stricture. The device may then be expanded to dilate the stricture.

The device may of course be used in any other desirable way.

1. A device for causing dilation of a tubular anatomy comprising:
a distal end and a proximate end;
an active element comprising a flexible portion expandable between a first, non expanded state, and a second, expanded state;
an activator for causing expansion of the flexible member; and
a pressure sensor operably connected to the flexible member.

2-34. (canceled)

35. The device according to claim 1, wherein the pressure sensor comprises a strain gauge.

36. The device according to claim 1, wherein the pressure sensor comprises a sensor skin mounted on an outer surface of the flexible portion.

37. The device according to claim 1, wherein the flexible portion comprises a plurality of elongate flexible members, each having a first end positioned towards the distal end of the device, and a second end positioned towards the proximate end of the device, the device further comprising a first connector connected to the first ends of the members, and a second connector connected to the second ends of the members.

38. The device according to claim 37, wherein the activator comprises a compressor for causing one of the connectors to move towards the other connector, thereby causing the elongate members to bow laterally.

39. The device according to claim 38, wherein the compressor exerts a constant pressure on the active element.

40. The device according to claim 38, wherein the compressor exerts stepped pressure on the active element.

41. The device according to claim 37, further comprising a plurality of flexible portions connected to one another.

42. The device according to claim 1, further comprising a passive outer shell enclosing the flexible portion.

43. The device according to claim 37, wherein the pressure sensor is positioned on one of the elongate flexible members.

44. The device according to claim 43, wherein the pressure sensor is defined by one or more cuts formed in the elongate flexible member.

45. The device according to claim 37, further comprising a plurality of pressure sensors, each of which is operably connected to an elongate flexible member.

46. The device according to claim 38, further comprising at least one stop for preventing over compression of the active element.

47. The device according to claim 46, further comprising a plurality of stops.

48. The device according to claim 47, wherein the stops comprise a plurality of grooves positioned along the length of the active element, and a moveable member in the form of a ring or disc.

49. The device according to claim 1, further comprising a screw thread extending along the length of the active element, and a moveable member moveable along the screw thread.

50. The device according to claim 37, wherein the first and second connectors are substantially disc shaped.

51. The device according to claim 37, wherein at least one of the connectors is formed integrally with the elongate members.

52. The device according to claim 37, wherein at least one of the connectors is formed separately from the elongate members.

53. The device according to claim 1, further comprising a first sleeve associated with the active element, the sleeve being mechanically driven.

54. The device according to claim 38, wherein the compressor comprises means for pulling one connector towards the other.

55. The device according to claim 38, wherein the compressor comprises a wire connected to the distal end of the active element.

56. The device according to claim 38, wherein the compressor comprises a screw.

57. The device according to claim 38, wherein the compressor comprises a threaded elongate member associated with the elongate member.

58. The device according to claim 1, wherein the activator is adapted to cause circumferential movement of the distal end of the device relative to the proximate end of the device.

59. The device according to claim 58, wherein the activator is adapted to cause circumferential rotation of the distal end in a first sense, and to cause circumferential rotation of the proximate end in an opposite sense.

60. The device according to claim 1, wherein the flexible portion comprises a balloon.

61. The device according to claim 60, further comprising a plurality of pockets formed on an outer surface of the balloon, each of the pockets containing a fluid, the device further comprising a pressure sensor operatively connected to each pocket.

62. The device according to claim 1, further comprising an outer sleeve extending over the active element.

63. The device according to claim 1, further comprising a cone formed at a distal end of the active element.

64. A device for causing dilation of a tubular anatomy comprising:
a distal end and a proximate end;
an active element comprising a flexible portion expandable between a first, non-expanded state, and a second, expanded state; and
an activator for causing expansion of the flexible portion,
wherein the flexible portion comprises a plurality of elongate flexible members, each having a first end positioned towards the distal end of the device, and a second end positioned towards the proximate end of the device, and
wherein the activator is adapted to cause circumferential movement of the distal end of the device relative to the proximate end of the device.

65. A method of dilating a tubular anatomy, the method comprising:
inserting a device for causing dilation of a tubular anatomy into the tubular anatomy to be dilated, wherein the device comprises:
an active element comprising a flexible portion expandable between a first, non-expanded state, and a second, expanded state,
an activator for causing expansion of the flexible portion, and
a pressure sensor operably connected to the flexible portion;
causing expansion of the flexible portion;
measuring pressure within the tubular anatomy;
collapsing the device; and
removing the device from the tubular Anatomy.
